(19) United States
(12) Patent Application Publication
(10) Pub. No.: US 2003/0083239 A1
(43) Pub. Date: May 1, 2003

(54) Chemokine receptor CCR3 antagonists

(76) Inventor: Bassam Damaj, San Diego, CA (US)

Correspondence Address: TRASK BRITT, P.O. BOX 2550, SALT LAKE CITY, UT, 84110, US

(21) Appl. No.: 10/202,615

(22) Filed: Jul. 23, 2002

Related U.S. Application Data
(63) Continuation of application No. 09/995,015, filed on Nov. 27, 2001.
Application No. 09/995,015 is a continuation of application No. PCT/CA00/00601, filed on May 23, 2000.
(60) Provisional application No. 60/136,377, filed on May 27, 1999.

(51) Int. Cl. A61K 38/12; A61K 38/08; C07K 7/64; C07K 7/06

(52) U.S. Cl. 514/9; 514/17; 530/329; 530/317

(57) ABSTRACT

The present invention relates to antagonists of chemokine receptor CCR3 and to a method of treating a subject with a disease associated with aberrant leukocyte recruitment and/or activation. The antagonist is a hexapeptide capable of inhibiting a beta-chemokine from binding to a CCR3 receptor. The method includes administering to a subject a therapeutically effective amount of a hexapeptide antagonist of chemokine receptor CCR3. The hexapeptide antagonist binds between a beta-chemokine and the receptor CC3 preventing a beta-chemokine from binding to the receptor CCR3.

### CROSS-REFERENCE TO RELATED APPLICATIONS

This application is a continuation of co-pending U.S. patent application Ser. No. 09/995,015, filed Nov. 27,2001, which was a continuation of co-pending International Patent Application No. PCT/CA00/00601 filed on May 23, 2000 designating the United States of America (International Publication No. WO 00/73327 published on Dec. 7, 2000), the contents of the entirety of which are incorporated by this reference, International Patent Application No. PCT/CA00/00601 itself claims priority to U.S. Provisional application No. 60/136,377 filed May 27, 1999.

### TECHNICAL FIELD

The present invention relates to antagonists of chemokine receptor CCR3 and to a method of treating a subject with a disease associated with aberrant leukocyte recruitment and/or activation.

### BACKGROUND

Chemokines are a family of proteins produced in many different tissues. The expression of chemokines is increased in response to infection or very early stages of inflammation. Chemokine receptors are members of the G protein-coupled family of receptors located on the outer membrane of the cell and translate a variety of signals from the outside to the inside of the cell. Chemokines bind to these chemokine receptors and activate leukocytes, causing them to migrate toward the source of the chemokine molecule (that is, movement out of the bloodstream and into tissues). Each subset of leukocytes (for example, eosinophils, monocytes, lymphocytes and neutrophils) has distinct types of chemokine receptors that respond to only certain chemokines. Through this discriminating mechanism, the body can control and selectively recruit certain types of leukocytes to mediate an inflammatory process. In addition, certain viruses, such as HIV-1, may use chemokine receptors as a homing mechanism to bind to and infect leukocytes.

Three classes of chemokines have been defined by the arrangement of the conserved cysteine (C) residues of the mature proteins: the CXC or α chemokines that have one amino acid residue separating the first two conserved cysteine residues; the CC or β chemokines in which the first two conserved cysteine residues are adjacent; the C or γ chemokines which lack two (the first and third) of the four conserved cysteine residues.

Within the CXC subfamily, the chemokines can be further divided into two groups. One group of the CXC chemokines has the characteristic three amino acid sequence ELR (glutamic acid-leucine-arginine) motif immediately preceding the first cysteine residue near the amino terminus. A second group of CXC chemokines lacks such an ELR domain. The CXC chemokines with the ELR domain (including IL-8, GROα/β/g, mouse KC, mouse MIP-2, ENA-78, GCP-2, PBP/CTAPIII/β-TG/NAP-2) act primarily on neutrophils as chemoattractants and activators, inducing neutrophil degranulation with release of myeloperoxidase and other enzymes. The CXC chemokines without the ELR domain (e.g., IP-10/mouse CRG, Mig, PBSF/SDF-1, PF4), the CC chemokines (e.g., MIP-1α, MIP-1b, RANTES, MCP-1/2/3/4/mouse JE/mouse MARC, eotaxin, I-309/TCA3, HCC-1, C10), and the C chemokines (e.g., lymphotactin), chemoattract and activate monocytes, dendritic cells, T-lymphocytes, natural killer cells, B-lymphocytes, basophils, and eosinophils.

In addition to their roles in regulating leukocyte recruitment and trafficking, certain chemokines have been reported to act on hematopoietic progenitor cells and on non-leukocytic cells such as fibroblasts, smooth muscle cells, keratinocytes and melanoma cell lines. Other chemokines have also been implicated as playing a role in wound healing, in angiogenesis and in viral infection. In in vitro assays, chemokines have overlapping and redundant functions. It remains to be determined to what extent the various chemokines have unique roles in vivo. To date, over 20 chemokines have been cloned and characterized. Additional chemokines have also turned up in various cloning strategies. The genes for all CC chemokines have been found to cluster on human chromosome 17q and mouse chromosome 11. With the exception of PBSF/SDF-1, all CXC chemokines genes have been found to cluster on human chromosome 4q. The human PBSF/SDF-1 gene and the gene for the C chemokine lymphotactin have been localized to human chromosome 10q and 1, respectively. The clustering of chemokine genes suggests that many cytokine family members arose through gene duplication and subsequent divergence.

Chemokines bind to heparin and glycosaminoglycans on cell surface proteoglycans. The immobilization of chemokines by cell surface proteoglycans or components of the extracellular matrix is thought to be important for the maintenance of the chemokine gradient needed for leukocyte activation and diapedesis and migration into tissue spaces.

Chemokine Receptors

Chemokines mediate their activities by binding to target cell surface chemokine receptors that belong to the large family of G protein-coupled, seven transmembrane (7 TM) domain receptors (also called serpentine receptors). Based on the receptor nomenclature established at the 1996 Gordon Research Conference on chemotactic cytokines, the chemokine receptors that bind CXC chemokines are designated CXCRs and the receptors that bind CC chemokines are designated CCRs.

Leukocytes have generally been found to express more than one receptor type. The various CXCRs and CCRs are known to exhibit overlapping ligand specificities. CCR-3 is a high affinity receptor for eotaxin/2, an eosinophil and a Th2 specific chemoattactant. In humans, CCR3 was found to be expressed exclusively on eosinophils and some clones of the Th2 cells. Eosinophils play prominent roles in a variety of atopic conditions including allergic rhinitis, dermatitis, conjunctivitis, and particularly bronchial asthma.

Elsner (Eur. J. Immun., 27:11, 2892-2898, 1997) describes Met-Rantes, a −9 kDa peptide, that is a modified version of the original chemokine Rantes where an extension has been added to the N-terminal of the peptide.

In Heath et al. (J. Clin. Invest., 99:2, 178-184, 1997), the authors describe the antagonistic activities of an antibody to the CCR3 receptor. A monoclonal antibody, mAB 7B 11, blocked chemokine binding to CCR3 and its subsequent functions.

Thus, it would be highly desirable to be identify and provide antagonists of the interaction between C-C chemokine receptor 3 and its ligands, including Eotaxin. Eotaxin 2, MCP-3, MCP-5 and RANTES.

### DISCLOSURE OF THE INVENTION

One embodiment of the present invention overcomes the drawbacks inherent in the prior art by providing new methods of modulating and inhibiting the action of a specific subset of CC chemokines through the CCR3 receptor.

Another aspect of the present invention provides a small molecular weight hexapeptide, that is an antagonist of chemokine receptor CCR3 function and inhibits leukocyte activation and/or recruitment. The hexapeptides can further inhibit beta-chemokine from binding to a CCR3 receptor. The hexapeptide of the present invention preferably has the amino acid sequence CRCAAR (SEQ ID NO: 1) or CACWWA (SEQ ID NO: 2). The hexapeptide is preferably at least acylated at its N-terminus or amidated at the C-terminus. Further, the hexapeptide may be a cyclic peptide or a reverse peptide.

The beta-chemokine is preferably selected from the group consisting of eotaxin, eotaxin-2, MCP-3, MCP-5, RANTES.

Still in accordance with the present invention, there is provided a method for treating a patient affected with a disease associated with aberrant leukocyte recruitment, activation or both. The method comprises the step of administering to the patient a therapeutically effective amount of an hexapeptide antagonist of chemokine receptor CCR3. The hexapeptide antagonist binds between a beta-chemokine and the receptor CCR3. The hexapeptide antagonist used in the method of the present invention preferably has a sequence as set forth in SEQ ID NO: 1 or SEQ ID NO: 2.

The hexapeptide antagonist is preferably is at least acylated at its N-terminus or amidated at the C-terminus, and may be a cyclic peptide or a reverse peptide

Further in accordance with the present invention, there is provided a method for inhibiting a beta-chemokine from binding to chemokine receptor CCR3. The method comprises the step of contacting a cell having the chemokine receptor CCR3 with an hexapeptide having a sequence as set forth in SEQ ID NO: 1 or SEQ ID NO: 2. The hexapeptide inhibits the beta-chemokine from binding to the receptor CCR3.

Also in accordance with the present invention, there is provided an hexapeptide as described above for use in treating a disease associated with aberrant leukocyte recruitment, activation or both.

In accordance with the present invention, there is also provided the use of an hexapeptide as defined above for the manufacture of a medicament for treating a disease associated with aberrant leukocyte recruitment, activation or both.

The present invention further provides a pharmaceutical composition comprising an hexapeptide as defined above, in combination with a pharmaceutically acceptable carrier.

By an antagonist of chemokine receptor CCR3 function, it is meant a molecule which can inhibit the binding of one or more chemokines, including C-C chemokines such as Eotaxin, Eotaxin 2, MCP-3, MCP-5, and RANTES, to chemokine receptor CCR3 on leukocytes and/or other cell types. As a consequence, processes and cellular responses mediated by chemokine receptors can be inhibited with these small organic molecules.

The antagonists of the present invention are useful for inhibiting harmful inflammatory processes “triggered” by receptor ligand interaction, as well as valuable tools for the investigation of receptor-ligand interactions.

By the term peptide, hexapeptide or antagonist, it is meant to include the peptide, hexapeptide or antagonist itself, as well as any physiologically acceptable salts thereof, or any chemically modification made thereto, which would be apparent or known to a person of ordinary skill in the art.

### BRIEF DESCRIPTION OF THE DRAWINGS

FIG. 1 is an illustration of the total binding of ¹²⁵I-eotaxin to CCR3 on human eosinophils in presence of different concentrations of an hexapeptide according to one embodiment of the present invention;

FIG. 2 is an illustration of the intracellular calcium mobilization in response to eotaxin with different concentration of the hexapeptide used for FIG. 1;

FIG. 3 is an illustration of the total number of cells migrating in response to eotaxin with different concentrations of the hexapeptide used for FIG. 1;

FIG. 4 is an illustration of the specificity of the hexapeptide used for FIG. 1 at 10 μM to bind to different chemokine receptors (CXCR1, 2, and CXCR4); and

FIG. 5 is an illustration of the cytotoxicity of different concentration of the hexapeptide used for FIG. 1, in human eosinophils.

### DETAILED DESCRIPTION OF THE INVENTION

In accordance with one embodiment of the invention, there is provided small molecular weight hexapeptides, which are antagonists of chemokine receptor CCR3 function. Accordingly, processes or cellular responses mediated by the binding of a chemokine to a receptor can be inhibited (reduced or prevented, in whole or in part), including leukocyte migration, integrin activation, transient increases in the concentration of intracellular free calcium ([Ca⁺⁺]ᵢ), and/or granule release of pro-inflammatory mediators.

The invention further relates to a method of treatment, including prophylactic and therapeutic treatments, of a disease associated with aberrant leukocyte recruitment and/or activation, including chronic inflammatory disorders characterized by the presence of Eotaxin, Eotaxin 2, MCP-3, MCP-5, and RATES responsive T cells, epithelial cells and/or eosinophils, including but not limited to diseases such as arthritis, psoriasis, multiple sclerosis, inflammatory bowel diseases such as ulcerative colitis and Crohn's disease, as well as allergies and asthma. Other diseases associated with aberrant leukocyte recruitment and/or activation which can be treated (including prophylactic treatments) with the methods disclosed herein are inflammatory diseases associated with Human Immunodeficiency Virus (HIV) infection, e.g., AIDS associated encephalitis, AIDS related maculopapular skin eruption, AIDS related interstitial pneumonia, AIDS related enteropathy, AIDS related periportal hepatic inflammation and AIDS related glomerulo nephritis. The method comprises administering to a subject a therapeutically effective amount of a compound (i.e., one or more compounds) which inhibits chemokine receptor CCR3 function, inhibits the binding of a chemokine to leukocytes and/or other cell types, and/or which inhibits leukocyte migration to, and/or activation at, sites of inflammation. According to the method, chemokine-mediated chemotaxis and/or activation of pro-inflammatory cells bearing receptors for chemokines can be inhibited. As used herein, “pro-inflammatory cells” includes but is not limited to leukocytes, since chemokine receptors may be expressed on other cell types, such as neurons and epithelial cells.

In a preferred embodiment of the present invention, the antagonist of chemokine receptor CCR3 function has the structural formula Ac-CRCAAR-NH2 (SEQ ID NO: 1).

In another preferred embodiment of the present invention, the antagonist of chemokine receptor CCR3 function has the structural formula Ac-CACWWA-NH2 (SEQ ID NO: 2).

Biological Functions Equivalents

Certain biological functional equivalents of the peptides are contemplated within the scope of this invention. The concept of biologically functional equivalent amino acids is well known to those in the art, and is embodied in the knowledge that modifications and changes may be made in the structure of a protein or peptide and still obtain a molecule having like or otherwise desirable characteristics.

However, it is also well understood by skilled artisans that, inherent in the definition of a biologically functional equivalent protein or peptide, is the concept that there is a limit to the number of changes that may be made within a defined portion of the molecule and still result in a molecule with an acceptable level of equivalent biological activity and that key active site or structurally vital residues cannot be exchanged. Biological functional equivalent peptides are therefore defined herein as those peptides in which certain, not most of all or all, of the amino acids may be substituted. In particular, where hexamer or heptamer peptides are concerned, it is contemplated that only about two, or more preferably, a single amino acid change would be made within a given peptide. Of course, a plurality of distinct peptides with different substitutions may be easily be made and used in accordance with the invention.

In regard to changing a limited number of residues within a peptide, it is known that certain amino acids may be substituted for other amino acids without appreciable loss of functions, as may be measured by the interactive binding capacity for structures such as receptors and cells, or the ability to compete with other molecules for binding to specific sites. Since it is the interactive and competitive capacity of a protein or peptide that defines its biological functional activity, certain amino acid substitutions can be made in a peptide sequence (or, of course its underlying DNA coding sequence) and nevertheless obtain a peptide with like, or even improved properties.

Pharmaceutical Formulations

The peptides and compositions of the invention may be used for treating a variety of diseases and disorders in which CCR3 chemokine receptor or eosinophils are involved or in which there is an appropriate or increased inflammatory response.

As the invention may be employed to treat bronchial asthma in various clinical settings, many types of pharmaceutical peptide formulation are contemplated. Therapeutic or pharmacological compositions of the present invention will generally comprise a therapeutically effective amount of a relatively small chemokine or chemokine-inhibiting-peptide or peptides, dissolved or dispersed in a pharmaceutically acceptable medium. The phrase “pharmaceutically acceptable” refers to molecular entities and compositions that do not produce an allergic, toxin or otherwise adverse reaction when administered to a human. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated.

Supplementary active ingredients can also be incorporated into the therapeutic compositions of the present invention. For example, eotaxin can also be combined with eosinophil-inhibitory-peptides, IFN-g, oxygen radical scavengers and the like, to create peptide cocktails for treatment. The preparation of pharmaceutical or pharmacological compositions containing eotaxin and an eosinophil-inhibiting-peptide or peptides, including dextrorotatory peptides, as an active ingredients will be known to those of skill in the art in light of the present disclosure. If desired, such compositions may be prepared as injectables, either as liquid solutions or suspensions; solid forms suitable for solution in, or suspension in, liquid prior to injection; as tablets or other solids for oral administration; as time release capsules; or in any other form currently used.

Solutions of the active peptides are compounds as free base or pharmacologically acceptable salts can be prepared in water suitable mixed with a surfactant, such as hydroxypropylcellulose. Dispersions can also be prepared in glycerol, liquid polyethylene glycols, and mixtures thereof and oils. Under ordinary conditions of storage and use, these preparations contain a preservative to prevent growth of microorganisms.

Sterile solutions suitable for injection are contemplated to be useful in treating various diseases and may be administered into blood stream or into the precise site of the inflammation. The pharmaceutical forms suitable for injectable use include sterile aqueous solutions or dispersions and sterile powders for the extemporaneous preparation of sterile injectable solutions or dispersions. In all cases the form must be sterile and must be fluid to the extend that easy syringability exists. It must be stable under the conditions of manufacture and storage and must be preserved against the contaminating action of microorganisms, such as bacteria and fungi.

A peptide can be formulated into a composition in a neutral or salt form. Pharmaceutically acceptable salts, include the acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxy groups can also be derived from inorganic bases such as, for example, sodium, potassium, ammonium, calcium, or ferric hydroxides, and such organic bases as isopropylamine, trimethylamine, histidine, procaine, and the like.

The carrier can also be a solvent or dispersion medium containing, for example, water ethanol, polyol (for example, glycerol, propylene glycol, and liquid polyethylene glycol, and the like), suitable mixtures thereof, and vegetable oils. The proper fluidity can be maintained, for example by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example aluminum monostearate and gelatin. Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of sterile injectable solutions, the preferred methods of preparation are vacuum drying and freeze-drying techniques, which yield a powder of the active ingredient, plus any additional desired ingredient from a previously sterile-filtered solution thereof.

The preparation of more, or highly, concentrated solutions for intramuscular injection is also contemplated. In this regard, the use of DMSO as solvent is preferred as this will result in extremely rapid penetration, delivering high concentration of the active peptide, peptides or agents to a small area.

Upon formulation, therapeutics will be administered in a manner compatible with dosage formulation, and in such amount as is pharmacologically effective. The formulations are easily administered in a variety of dosage forms, such as the type of injectable solutions described above, but drug release capsules and the like can also be employed.

A minimal volume of a composition required to disperse the peptide is typically utilized. Suitable regimes for administration are also variable, but would be typified by initially administering the compound and monitoring the results and then giving further controlled--doses at further intervals. For example, for parental administration, a suitable buffered, and if necessary, isotonic aqueous solution would be prepared and used for intravenous, intramuscular, subcutaneous or even intraperitoneal administration. One dosage could be dissolved in 1 ml of isotonic NaCl solution and either added to 1000 ml of hypodermoclysis fluid or injected at the proposed site of infusion.

In certain embodiments, active compounds may be administered orally. This is contemplated for agents who are generally resistant, or have been rendered resistant, to proteolysis by digestive enzymes. Such compounds are contemplated to include dextrorotatory peptides; chemically designed or modified agents; and peptide and liposomal formulation in time-release capsules to avoid peptidase degradation.

Oral formulations may include compounds in combination with an inert diluent or an assimilable edible carrier; those enclosed in hard or soft shell gelatin capsules; those compressed into tablets; or those incorporated directly with the food of the diet. For oral therapeutic administration, the active compounds may be incorporated with excipients, syrups, wafers, and the like. Such compositions and preparations should generally contain at least 0.1% of active compound. The percentage of the compositions and preparations may, of course, be varied and may conveniently be between about 2 to 60% of the weight of the unit. The amount of active compounds in such therapeutically useful compositions is such that a suitable dosage will be obtained.

Tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. Syrup of elixir may contain the active compound, sucrose as a sweetening agent methyl and propylparabens as preservatives, a dye and flavoring, such as cherry or orange flavor. Of course, any material used in preparing any dosage unit should be pharmaceutically pure and substantially non-toxic in the amounts employed. In addition, the active compounds may be incorporated into sustained-release preparation and formulation.

The following examples are included to demonstrate preferred embodiment of the invention. It should be appreciated by those of skill in the art that the techniques disclosed in the examples which follow represent techniques discovered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The present invention will be more readily understood by referring to the following examples, which are given to illustrate the invention rather than to limit its scope.

### EXAMPLE I

### Inhibition of Eotaxin Binding to CCR3 Receptors by SC004411

A series of studies was first carried out to determine whether hexamer peptides of the sequence CRCAAR (SEQ ID NO: 1), also referred to as peptide SC004411, would act as inhibitors of IL-8. The assays of the initial screen are based upon determining the ability of a giving peptide to inhibit the binding of eotaxin to CCR3 on human eosinophils.

Human Eosinophil Preparation

Human eosinophils were prepared by isolation from the blood of donor individuals with high levels of circulating blood eosinophils (5-17%) by combining density gradient centrifugation and negative selection with anti-CD16 magnetic beads. Briefly, the granulocyte fraction from the Percoll centrifugation was incubated with CD16 micro beads (miniMACS™, separation unit) for 30 minutes. Cells were then passed through a MACS™ column (Miltenyi Biotec, Inc., Auburn, Calif.) and eosinophils were collected in the flow through. Eosinophil purity was >98% as determined by analysis of Diff-Quik™ (Baxter) stained cytocentrifugation preparations by light microscopy.

Peptides

All peptides were synthesized using the tBOC for protection of the α-amino group. All synthetic peptides were purified on high performance liquid chromatography (HPLC) using a preparative C18 reverse phase column™. Peptides were eluted using a gradient from 0 1% trifluoroacetic acid (TFA) to 80% acetonitrile in 0.1% TFA. The composition of the peptides was confirmed by amino acid analysis and sequencing.

Binding Assays

Twenty five thousands cells suspended in 100 μl (25,000 cells/100 μl) were incubated with 0.1 to 0.2 nM ¹²⁵I-labeled Eotaxin with or without unlabeled competitor (Eotaxin) or various concentrations of the hexamer peptide of SEQ ID NO: 1 (SC004411). The binding reactions were performed in 100 μl of a binding buffer consisting of 10 mM HEPES pH 7.2, 1 mM CaCl₂ 5 mM MgCl₂, and 0.5% BSA (bovine serum albumin), for 60 min at room temperature. The binding reactions were terminated by harvesting the cells by rapid filtration through glass fiber filters (GF/B™ or GF/C™, Packard) which were presoaked in 0.3% polyethyleneimine. The filters were rinsed with approximately 600. μl of binding buffer containing 0.5 M NaCl, dried, and the amount of bound radioactivity was determined by scintillation counting in a Topcount™ betaplate counter.

Ligand Binding Assay

¹²⁵I RANTES and ¹²⁵I-EOTAXIN were purchased from DuPont-NEN (Boston, Mass.) with a specific activity of 2,200 Ci/mM.

Chemokine binding to the target cells, human eosinophils and 293/CCR3 cells, was carried out using a modification of a method previously reported. Cells were washed once in PBS and resuspended in binding buffer (50 mM HEPES, 1 mM CaCl₂, 5 mM MgCl₂, and 0.5% BSA) at a concentration of 1×10⁷/mL. Aliquots of 50 μL (5×10⁵ cells) were dispensed into microfuge tubes, followed by the addition of cold and radiolabeled chemokines. The final reaction volume was 200 μL. Nonspecific binding was determined by incubating cells with radiolabeled chemokines in the presence of increasing amounts of (250-500 nM) of cold chemokine. After 60-min incubation, at room temperature, the cells were washed three times with 1 ml of binding buffer plus 0.5 M NaCl. Cell pellets were then counted. All experiments were carried out using duplicates and repeated at least three times. Curve fit was calculated by Kaleidagraph™ software (Synergy Software, Reading, Pa.). Inhibition of binding was assessed by the addition of test inhibitor compound at the indicated concentrations, and incubation for 30 min prior to addition of the chemokine as above.

Results

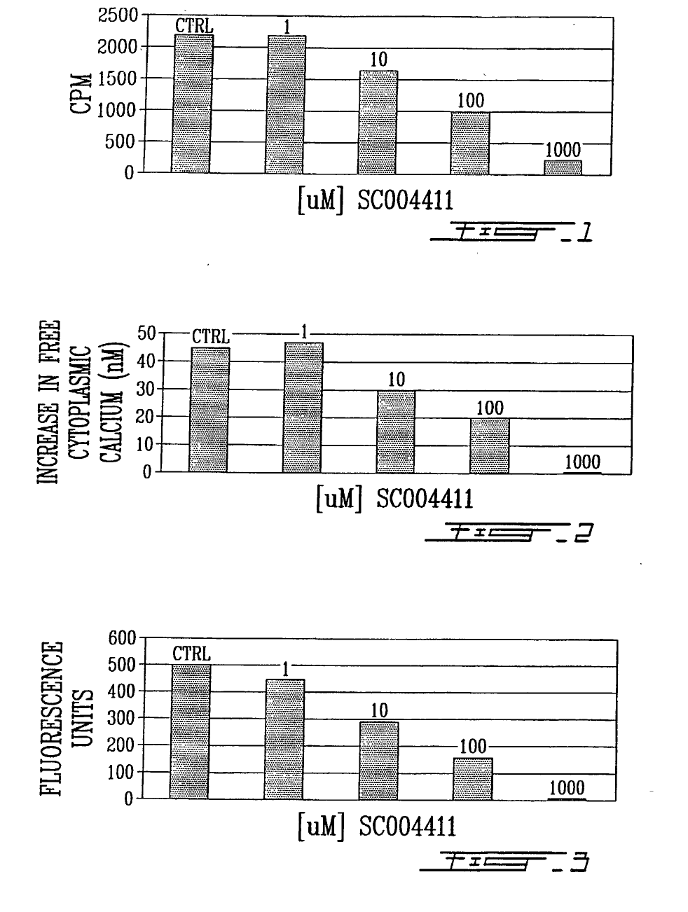

FIG. 1 shows the inhibition of ¹²⁵I-eotaxin binding to human eosinophils by compound SC004411 having SEQ ID NO: 1. The compound effectively competed with ¹²⁵I-eotaxin to bind to CCR3 with an IC₅₀ close to 40 μM. Labeled eotaxin was used at 0.3 nM. The data are representative of three independent experiments.

### EXAMPLE II

### Inhibition of Eotaxin-Induced Calcium Mobilization in Eosinophils by SC004411

Calcium Mobilization

Intracellular free calcium was measured using the fluorescent probe Fluo-3. Cell suspensions (10⁷ cells/ml) were loaded with Fluo-3/AM (1 μM, 30 minutes at 37° C), washed, resuspended at 2×10⁶ cells/ml in Hanks' balanced salt solution (HBSS) and their fluorescence monitored in a spectrofluorometer (SLM 8000, SLM-Aminco, Champagne, Ill., USA) (excitation and emission wavelengths, 500 and 530 nm, respectively) The internal calcium concentrations were calculated as described in Tsien et al. (J. Cell. Biol. 94: 3325-3334, 1982). Each assay was individually calibrated.

Results

FIG. 2 shows the inhibition of eotaxin-induced calcium mobilization of human eosinophils by compound SC004411, having SEQ ID NO: 1, in human eosinophils. The compound effectively blocked eotaxin-induced calcium mobilization in human eosinophils with a functional IC₅₀ close to 40 μM. Eotaxin was used at 10 nM. The peak response induced by eotaxin is shown. The data are representative of three independent experiments.

### EXAMPLE III

### Inhibition of Eotaxin-Induced Eosinophil Chemotaxis by SC004411

Chemotaxis

Leukocyte chemotaxis was assessed on eosinophils, using a modification of a transendothelial assay. The endothelial cells used in this assay were the endothelial cell line, ECV 304, obtained from the European collection of Animal Cell Cultures (Porton Downs, Salisbury, U.K.). Endothelial cells were cultured on 6.5 mm diameter Transwell™ culture inserts (Costar Corp., Cambridge, Mass.) with 3.0 μm pore size. The assay media consisted of equal parts RPMI 1640 and M199 with 0.5% BSA. Two hours before the assay, 2×10⁵ ECV 304 cells were plated onto each insert of the 24 wells Transwell™ chemotaxis plate and incubated at 37° C. Chemotactic factors (diluted in assay medium) were added to the 24-well tissue culture plates in a final volume of 600 μL. Endothelial-coated Transwells™ were inserted into each well and 106 cells of the leukocyte type being studied were added to the top chamber in a final volume of 100 μL of assay medium. The plate was then incubated at 37° C. in 5% CO₂/95% air for 1-2 h. The cells that had migrated to the bottom chamber were counted using flow cytometry. Five hundreds (500) μL of the cell suspension from the lower chamber was placed in a tube and relative counts were obtained for a set period of time of 30 seconds. This counting method was found to be highly reproducible and enabled gating on the leukocytes and the exclusion of debris or other cells. Counts obtained by this method matched closely those obtained by counting with a microscope. Assays evaluating chemotaxis inhibitors were performed in the same way as control experiments above, except that inhibitor solutions, in assay media containing up to 1% of DMSO co-solvent, were added to both the top and bottom chambers prior to addition of the cells. Inhibitor potency was determined by comparison of cell numbers migrated to the bottom chamber, with or without inhibitor. Control wells contained equivalent amounts of DMSO, but no inhibitor.

Results

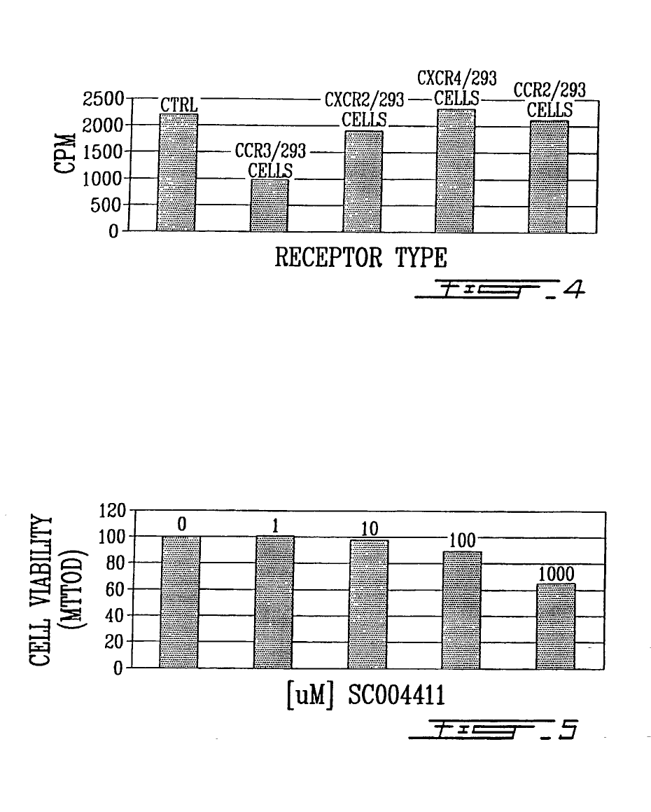

FIG. 3 shows the inhibition of eotaxin-induced chemotaxis of human eosinophils by compound SC004411, having SEQ ID NO: 1. Compound SC004411 effectively blocked the eotaxin-induced chemotaxis of human eosinophils with a functional IC₅₀ close to 20 μM. Eotaxin was used at 10 nM. The peak response induced by eotaxin is shown. The data are representative of three independent experiments.

### EXAMPLE IV

### Specificity of SC004411 to CCR3 Receptors

FIG. 4 shows the binding specificity of compound SC004411, having SEQ ID NO: 1, to the CCR3 receptor against other chemokine receptors by ¹²⁵I-eotaxin binding. The compound was used in the binding assay to compete with CXCR4, CXCR2, CCR2, and CCR3 transfected in 293 cells. As shown in FIG. 4, compound SC004411 specifically inhibited eotaxin-binding to CCR3 but not to CCR2, CXCR2 or CXCR4. The data are representative of three independent experiments.

### EXAMPLE V

### Cytotoxicity of SC004411

Cytotoxicity

To exclude the possible contribution of cytotoxicity of the hexapeptides to the inhibition of eosinophil function, ⁵¹Cr-labeled cells were cultured in presence of the peptide for the indicated time. The peptide did not cause cell lysis of human eosinophils. Measurement of Trypan Blue exclusion also indicated that the peptide was not toxic to the cells.

Results

In FIG. 5, cell viability in presence of SC004411 was assayed with MTT incorporation. As shown in FIG. 5, compound SC004411 starts to be toxic to human eosinophils after 24 hours at concentration above 100 μM, that is above the IC₅₀ concentration). The data are representative of three independent experiments.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A hexapeptide for inhibiting a beta-chemokine from binding to a CCR3 receptor, said hexapeptide having a sequence as set forth in SEQ ID NO: 1 or SEQ ID NO: 2.

2. The hexapeptide of claim 1, wherein said beta-chemokine is selected from the group consisting of eotaxin, eotaxin-2, MCP-3, MCP-5 and RANTES.

3. The hexapeptide of claim 1, wherein said hexapeptide is at least acylated at its N-terminus or amidated at the C-terminus.

4. The hexapeptide of claim 1, wherein said hexapeptide is a cyclic peptide.

5. The hexapeptide of claim 1, wherein said hexapeptide is a reverse peptide.

6. A method of treating a subject affected with a disease associated with aberrant leukocyte recruitment, aberrant leukocyte activation or both, said method comprising administering a therapeutically effective amount of a hexapeptide antagonist of a chemokine receptor CCR3 to said subject, said hexapeptide antagonist binding between a beta-chemokine and the chemokine receptor CCR3 and having a sequence as set forth in SEQ ID NO: 1 or SEQ ID NO: 2.

7. The method according to claim 6, wherein said beta-chemokine is selected from the group consisting of eotaxin, eotaxin-2, MCP-3, MCP-5 and RANTES.

8. The method according to claim 6, wherein said hexapeptide antagonist is at least acylated at its N-terminus or amidated at the C-terminus.

9. The method according to claim 6, wherein said hexapeptide is a cyclic peptide.

10. The method according to claim 6, wherein said hexapeptide is a reverse peptide.

11. A method for inhibiting a beta-chemokine from binding to a chemokine receptor CCR3, said method comprising delivering to a cell having the chemokine receptor CCR3 a hexapeptide having a sequence as set forth in SEQ ID NO: 1 or SEQ ID NO: 2, said hexapeptide inhibiting the beta-chemokine from binding to the chemokine receptor CCR3.

12. The method according to claim 11, wherein said beta-chemokine is selected from the group consisting of eotaxin, eotaxin-2, MCP-3, MCP-5 and RANTES.

13. The method according to claim 1 1, wherein said hexapeptide is at least acylated at its N-terminus or amidated at the C-terminus.

14. The method according to claim 11, wherein said hexapeptide is a cyclic peptide.

15. The method according to claim 1 1, wherein said hexapeptide is a reverse peptide.

16. A method for treating a disease associated with aberrant leukocyte recruitment, aberrant leukocyte activation or both, said method comprising inhibiting a beta-chemokine from binding to a CCR3 receptor by administering the hexapeptide of clam 1 to a subject having said disease.

17. The method according to claim 16, wherein said hexapeptide inhibits a beta-chemokine from binding to a receptor CCR3.

18. A method of manufacturing a medicament for treating a disease associated with aberrant leukocyte recruitment, activation or both, said method comprising providing the hexapeptide of clam 1 together with a pharmaceutically acceptable carrier.

19. The method according to claim 18, wherein said hexapeptide inhibits a beta-chemokine from binding to a receptor CCR3.

20. A pharmaceutical composition comprising a hexapeptide as defined in claim 1, in combination with a pharmaceutically acceptable carrier.

21. The pharmaceutical composition of claim 20, wherein said hexapeptide inhibits a beta-chemokine from binding to a chemokine receptor CCR3.

22. A method for inhibiting a beta-chemokine from binding to a chemokine receptor CCR3 said method comprising introducing the hexapeptide of clam 1 to a cell including the chemokine receptor CCR3.


SEQUENCE LISTING (2 sequences)

SEQ ID NO: 1 - protein, 6 aa, Artificial Sequence
  Chemokine receptor CCR3 antagonists
CRCAAR

SEQ ID NO: 2 - protein, 6 aa, Artificial Sequence
  Chemokine receptor CCR3 antagonists
CACWWA